(19) United States
(12) Patent Application Publication
(10) Pub. No.: US 2012/0029358 A1
(43) Pub. Date: Feb. 2, 2012

(54) Three -Dimensional Ultrasound Systems, Methods, and Apparatuses

(75) Inventor: Shengtz Lin, Cupertino, CA (US)

(73) Assignee: SONOWISE, INC., San Jose, CA (US)

(21) Appl. No.: 13/253,402

(22) Filed: Oct. 5, 2011

Related U.S. Application Data
(63) Continuation-in-part of application No. 12/729,620, filed on Mar. 23, 2010.
Application No. 12/729,620 is a continuation of application No. 11/193,935, filed on Jul. 29, 2005, now Pat. No. 7,708,691.
(60) Provisional application No. 60/658,889, filed on Mar. 3, 2005.

(51) Int. Cl. A61B 8/14

(52) U.S. Cl. 600/447

(57) ABSTRACT

In part, the invention relates to an immersible ultrasound probe having a substantially cylindrical shape and a circular or elliptical cross-section. Typically, the circumference of the probe and the length of the cylinder define an inner surface upon which rows and/or columns of transducer are disposed. This surface can also be formed from panels or modules. The transducers can be formed in unitary substrate and electrical connected to a MEMs device and a multiplexer. The inner surface defines a cavity having at least one opening sized to receive a body object. The inner surface configured to receive acoustic signals while immersed in a fluid.

### CROSS REFERENCE TO RELATED APPLICATIONS

This application is a continuation in part application that claims priority to and the benefit of U.S. Provisional Application No. 60/658,889 filed Mar. 3, 2005, U.S. Non-provisional application Ser. No. 11/193,935, filed Jul. 29, 2005, now U.S. Pat. No. 7,708,691, and U.S. Non-provisional application Ser. No. 12/729,620, filed Mar. 23, 2010, the entire disclosures of each of which are hereby incorporated by reference herein for all purposes.

### FIELD OF THE INVENTION

This invention generally relates to systems and methods for carrying out a medical imaging process. More particularly, this invention relates to an ultrasonic imaging apparatus and method for tissue scanning to improve the accuracy and early detection of breast cancer through the image scanning and analyses processes.

### BACKGROUND OF THE INVENTION

Even though an early detection of cancer tissues through image scanning is very desirable to greatly improve the cure rates and although the general technologies for image scanning have made significant advancements, there are still technical difficulties and limitations faced by application of ultrasonic imaging for real time three dimensional (3D) breast scanning. Specifically, the accuracy of measurements is still not reliable due to several factors as will be further discussed below.

The mammogram scan is a preferred technique for screening of breast cancer according to the United State governmental health policy. This is mainly because of a relatively low cost and high efficiency to perform the mammogram scans. However, the accuracy of mammogram results is still questionable for women with dense breasts. Conventionally, ultrasound imaging can be used for the breast cancer screening application. U.S. Pat. No. 6,117,080, “Ultrasonic imaging apparatus and method for breast cancer diagnosis with the use of volume rendering”, (the '080 patent) describes one conventional system and method for ultrasound imaging for breast cancer screening. However, conventional ultrasound imaging is merely suitable as a complementary solution for breast cancer screening. This limitation is due to the fact that the result of ultrasound imaging is strongly dependent on the skill of the person conducting the scanning. Therefore, the data and the diagnostic results are not consistently reliable.

In a conventional ultrasound imaging for breast cancer screening, as described in the '080 patent, the breast is scanned by sliding the scanhead over the surface of the breast. The scanhead needs to be held in a constant vertical orientation so that the images are acquired from substantially parallel scan planes. Due to the variation in breast tissue thickness across the breast, the scanhead will generally move in a slight arc in the y direction as that shown in FIG. 1A according to the '080 patent's coordinate system, as the scanhead moves across the breast surface.

Further, when the clinician is performing the scanning, the process maintains a constant acoustic contact with the breast and that asserts a certain amount of pressure as the scanhead moves. The constant pressure thus slightly compresses the breast tissue beneath the scanhead that leads to degradation of the accuracy and quality of data obtained from the ultrasonic image scans. The '080 patent suggested a scanning of the breast by freehand in which the user has to move the scanhead at a constant rate so that the image planes are separated in the z dimension by a substantially uniform separation which requires a few trials with slow scanhead movement and more rapid scanhead movement so the user can arrive at a scanning speed which will produce the best images. This level of skill requires significant training and practice thus limit the usefulness and acceptance of data obtained from the full breast scanning due to concerns of variations of the scanning process that may heavily depend on the skill level of an image scan operator.

The '080 patent further suggested that the arc in the y direction mentioned above can be minimized with the breast flattened out somewhat when the patient is reclining and with a water bag in between the scanhead and the breast tissue which conforms to the contours of the breast and provides good acoustic coupling between a scanning surface and the breast. This method is time consuming and does not give a consistent result and is limiting in other ways. For example, the water bag is suitable only for scanning in a down direction. Therefore, any scanning that is desired in other directions for tomography would need to take place without the water bag. However, because the water bag compresses the breast, the scans that are performed with and without the water bag would not match well.

As shown in FIG. 1B, the '080 patent also suggested an external devices attached to the scanhead to assist in determining scanhead position during a scan. A linear movement-sensing device is mounted on the scanning surface of the scanning aid and the linear sensor in the housing provides signals indicative of the position of the scanhead by means of a cable, which connects to the ultrasound system through a connector. This method again does not address the problem of unreliable data obtained in a scan process that requires the scan head to have multi-dimensional movement during the scan.

For these reasons, a need still exists for those of ordinary skill in the art to provide an improved method and system for medical imaging. Specifically, it is desirable that the scanning system and methods are carried out without overheating the sample being imaged or taking too long to perform the scan. It is further desirable that the scan process is automated and standardized for 3D volume data acquisition such that human operations and potential errors and variations can be minimized.

### SUMMARY OF THE INVENTION

It is an object of the present invention to provide a body object scanning system by applying an ultrasonic scanning device transmitting an ultrasonic waves through a coupling medium and to receive a reflection from the body object for processing and constructing a three dimensional image of the body object using image data such as images of cross-sections or along planes of the body object. The speed of the scanning and image construction is improved because the transducer array can remained fixed and thus does not need to be translated in space relative to the body object.

In another aspect, the present invention provides an ultrasonic image generation tomographic system with an automatic scanning process that does not depend on human skill. Accordingly, standardized processes may be implemented to obtain reliable and consistent image scanning data and more accurately process and determine whether there are physical abnormalities, such as tumors, lesions, or other anomalies in or on the body object.

In another aspect, the invention relates to an ultrasonic image scanning system for scanning a body object having one or more tissue structures disposed therein. The system includes an immersible ultrasound probe having a substantially cylindrical shape, the probe comprising an inner surface defining a cavity having at least one opening sized to receive the body object, the inner surface configured to receive acoustic signals while immersed in a fluid; and a plurality of modules arranged to form the inner surface, each module comprising a high voltage multiplexer, and a plurality of ultrasonic transducers, wherein the plurality of ultrasonic transducers is arranged in a plurality of rows and columns, wherein each row is adjacent to at least another row. In one embodiment, the system includes a baffle layer positioned in each module behind the plurality of ultrasonic transducers to reduce an acoustic reverberation associated with ultrasonic image scanning. Further, each module of the plurality of modules can have substantially rectangular shape having a length that ranges from about 100 mm to about 300 mm and a width that ranges from about 30 mm to about 60 mm. The plurality of transducers per module can be at least 64 transducer elements. In one embodiment, each of the plurality of ultrasonic transducers comprises a material selected from the group consisting of Lead zirconate titanate, a ceramic PZT material, CMUT, PMUT, and a semiconductor. In one embodiment, each of the plurality of ultrasonic transducers is a MEMS device configured to produce substantially parallel beams on a per row basis and in electronic communication with at least one of the high voltage multiplexers.

In one embodiment, the high voltage multiplexer is in electrical communication with the probe and configured to route signals between the probe and an ultrasound system. The ultrasonic image scanning system can also include a computer aided diagnostic system for processing a set of two-dimensional or three-dimensional image scans to display tissue structural characteristic details of the body object and identify one or more tissue structures. Each module can include a flexible or rigid printed circuit board configured to support each of the ultrasonic transducers and orient them towards the body object. The system can include a container configured to receive the probe, the container comprising a port for filling the container with coupling solution.

In one embodiment, the probe is configured to prevent excessive tissue heating during a scan such that the temperature of the body object ranges from 37 degrees Celsius to about 42 degrees Celsius during a scan. The ultrasonic image scanning system can have a volume scan rate that ranges from about 0.5 volumes per second to about 50 volumes per second. The ultrasonic image scanning system can include a control system in electrical communication with the probe and configured to trigger frame capture and transmit a clock pulse to one of the MEMS or the multiplexer.

In yet another aspect, the invention relates to a method for ultrasonically scanning a body object having one or more tissue structures disposed therein. The method includes the steps of electronically addressing a first plurality of transducers arranged in substantially circular configuration using a first control signal; transmitting a first plurality of incident acoustic waves from the first plurality of transducers in response to the first control signal; receiving a first plurality of returning acoustic waves at the first plurality of transducers, the first plurality of returning acoustic waves reflected from a body object; electronically addressing a second plurality of transducers arranged in substantially circular configuration using a second control signal; transmitting a second plurality of incident acoustic waves from the second plurality of transducers in response to the second control signal; and receiving a second plurality of returning acoustic waves at the second plurality of transducers, the second plurality of returning acoustic waves reflected from the body object. The method can also include the steps of converting the first plurality of returning acoustic waves and the second plurality of returning acoustic waves into one or more electrical signals and generating an image of the body object using an ultrasound system and the one or more electrical signals. In one embodiment, the body object is a breast or any soft tissue and further comprising the step of tomographically rendering a three-dimensional image of the breast or any soft tissue and one or more tissue structures disposed therein. The method can include the step of heating the body object such that the change in a temperature of the body object is less than between about 37 and about 42 degrees Celsius. In one embodiment, the heating is performed by one or both of the first and second incident acoustic waves. The method can include generating a tomographic image of the body object using the first and second pluralities of returning acoustic waves. The method can include the step of displaying one or more tissue structures. In one embodiment, the steps of the method are performed within between about 1 second to about 10 seconds. The method can include the step of identifying a tissue structure as likely to be cancerous on a display. The method can include the step of identifying a tissue structure as likely to be benign on a display. In one embodiment, the first plurality of incident acoustic waves is arranged or propagated in a substantially parallel configuration. In one embodiment, the second plurality of incident acoustic waves is arranged in a substantially parallel configuration.

In yet another aspect, the present invention further provides an ultrasonic imaging scanning system that includes an array of ultrasonic sensors whereby physical movement of the sensing system is not required in the 360 degree viewing of the 2D image to further simplify the scanning processes and to improve the accuracy of the scanning results.

In yet another aspect, the present invention provides a method of demultiplexing and multiplexing the ultrasonic image scanning processes from a different viewing angle by alternately turning on the scanning sensors according to a time demultiplexed sequence and multiplexing the signals measured from the array of sensors for transmitting and processing the multiplexed signals to construct a three dimensional scan image of a body object. Improvement of accuracy is achieved by implementing signal processing algorithms in managing and processing these images from 360 degrees of viewing angles in X-Y dimension and different positions in Z axis.

In one embodiment, the invention relates to an ultrasonic image scanning system for scanning an organic object, such as body object, that includes a plurality of modules, each module comprising a high voltage (HV) multiplexer and a plurality of ultrasonic transducers, a container for containing a coupling medium surrounded by each of the high voltage multiplexers and the plurality of ultrasonic transducers, each module disposed immediately adjacent to the container for transmitting an ultrasonic signal to the organic object immersing or submerging directly in the coupling medium, whereby a simultaneous multiple direction scanning process may be carried out with or without physically contacting the organic object because of no moving parts involved; and the plurality of ultrasonic transducers further receiving echo signals from the organic object transmitted in the coupling medium wherein the ultrasonic signal transmitted to and the echo signal transmitted from the organic object are transmitted completely through and within the coupling medium for improving an accuracy of an ultrasonic image scan; and a baffle layer positioned in the module in the areas between each of the plurality of ultrasonic transducers to reduce an acoustic reverberation associated with the scanning.

In one embodiment, the invention relates to an ultrasonic image scanning system for scanning an organic object that includes a container for containing a coupling medium for transmitting an ultrasonic signal to the organic object disposed therein whereby a simultaneous multiple direction scanning process may be carried out with or without physically contacting the organic object because of no moving parts involved. The ultrasonic image scanning system further includes ultrasound transducers for transmitting the ultrasonic signal to the organic object through the coupling medium without asserting an image deforming pressure to the organic object. These transducers distributed substantially around a two-dimensional perimeter of the container and substantially at symmetrical angular positions at approximately equal divisions of 360 degrees over a two-dimensional perimeter of the container. The transducers are further movable over a vertical direction alone sidewalls of the container for a real time three dimensional (3D) image data acquisition. The container further includes sidewalls covered with a baffle layer for reducing an acoustic reverberation.

In one embodiment, the invention relates to an ultrasonic image scanning system wherein the ultrasound transducers are arranged on a same horizontal level surrounding the container for simplifying an image geometrical and signal transmission and echoing analysis based on a configuration of the same horizontal level. The ultrasound transducers can be distributed and substantially surrounding an entire perimeter of the container. The ultrasound transducers are distributed substantially at symmetrical angular positions at approximately equal divisions of 360 degrees over an entire perimeter of the container or surface area of the side of the container.

Further, the ultrasound transducers can be distributed substantially around an entire perimeter of the container wherein the transducers are further movable over a direction perpendicular to a 2D scan plane of the transducers along sidewalls of the container for a real time three dimensional (3D) image data acquisition. In one embodiment, the baffle layer has an irregular shape for acoustic absorption. In one embodiment, the high voltage (high voltage) semiconductor multiplexer integrated circuit (IC) is connected to an array of ultrasound probes for real time scanning and data acquisition. The coupling medium can further include de-bubbled fluid. The ultrasonic image scanning system can includes a wire phantom for carrying out a scan conversion geometric calibration. The ultrasonic image scanning system can further include an image data processor for performing a three-dimensional volume and/or surface rendering for representing a scanned image of the organic object. The ultrasonic image scanning system can further include a computer aided diagnostic (CAD) system for processing a set of image scan data for constructing and presenting tissue structural characteristic details of the organic object according to the set of image scan data.

The ultrasonic image scanning system can further include a dense breast screen device to prescreen a dense breast as an organic object for carrying out an image scan by the ultrasonic image scanning system. The ultrasound transducers can be distributed substantially with a uniform circular arrangement for acquiring an ultrasonic image vector in a constant arc distance for reconstructing an image of the organic object. The ultrasonic image scanning system can also include omni-directional high frequency ultrasound probes. The ultrasound transducers can be further distributed over an entire interior surface of the container with a sufficient number of ultrasound transducers to capture a three-dimensional image of the organic object without requiring a movement of the ultrasound transducers.

The ultrasonic image scanning system can also include a scan controller to automatically control an image scanning process and data acquisition by controlling the ultrasound transducers with a standard scanning procedure. The ultrasonic image scanning system can also include a high density miniature connector to transmit signals between the scan controller and the ultrasound transducers. The ultrasonic image scanning system can also include a high density miniature connector further comprising a high voltage semiconductor multiplex switch for controlling a real time signal routing. The ultrasound transducers can be controlled to move around to different angular positions of the container for transmitting the ultrasonic signal to the organic object through the coupling medium without deforming the organic object.

In one embodiment, the invention relates to a method for ultrasonically scanning an organic object includes filling a container with a coupling medium placing a plurality of modules comprising a plurality of ultrasonic transducers immediately around the container for transmitting an ultrasonic signal to and receiving echo signals from the organic object, each module further comprising a high voltage multiplexer; immersing directly in the coupling medium for carrying out a simultaneous multiple direction scanning process without physically contacting the organic object wherein the ultrasonic signals transmitted to and the echo signals transmitted from the organic object are transmitted completely through and within the coupling medium for improving an accuracy of an ultrasonic image scan; and providing a baffle layer within the module in the areas between each of the plurality of ultrasonic transducers to reduce an acoustic reverberation using the baffle layer. The method can further include arranging the plurality of ultrasound transducers on a same horizontal level surrounding the container for simplifying an image geometrical and signal transmission and echoing analysis based on a configuration of the same horizontal level.

In one embodiment, the method can further include distributing the plurality of ultrasound transducers to substantially surround an entire perimeter of the container. The method can further include distributing a plurality of ultrasound transducers substantially at symmetrical angular positions at approximately equal divisions of 360 degrees over an entire perimeter of the container. The method can further include distributing a plurality of ultrasound transducers substantially around an entire perimeter of the container wherein the transducers are further movable over a direction perpendicular to a 2D scan plane of the transducers along sidewalls of the container for a real time three dimensional (3D) image data acquisition. The method can further include distributing the plurality of ultrasonic transducers over an entire interior surface of the container with sufficient number of ultrasonic transducers to capture a three-dimensional image of the organic object without requiring a movement of the ultrasonic transducers.

In one embodiment, the invention relates to an ultrasonic image scanning system for scanning an organic object includes a container for containing a coupling medium for transmitting an ultrasonic signal to the organic object disposed therein, whereby a simultaneous multiple direction scanning process may be carried out without physically contacting the organic object, and the container comprising sidewalls; a plurality of modules, each module comprising a high voltage multiplexer and a plurality of ultrasonic transducers, the plurality of ultrasound transducers for transmitting the ultrasonic signal to the organic object through the coupling medium without asserting an image deforming pressure to the organic object, the modules distributed substantially around a two-dimensional perimeter of the container, the ultrasound transducers distributed substantially at symmetrical angular positions at approximately equal divisions of 360 degrees over the two-dimensional perimeter of the container, the transducers further movable over a vertical direction along sidewalls of the container for a real time three dimensional image data acquisition; and a baffle layer positioned in the areas between each of the plurality of ultrasonic transducers to reduce an acoustic reverberation associated with the scanning the organic object. The baffle layer can include rubber for acoustic energy absorption and the module can include a printed circuit board that supports each of the ultrasonic transducers and the high voltage multiplexer.

In one embodiment, the invention relates to an ultrasonic image scanning system for scanning an organic object includes a circular ultrasound probe having a circumference, the probe includes a plurality of modules arranged along the circumference, each module comprising a high voltage multiplexer and a plurality of ultrasonic transducers; and a baffle layer positioned in each module in areas surrounding each of the plurality of ultrasonic transducers to reduce an acoustic reverberation associated with ultrasonic image scanning.

In one embodiment, the ultrasound transducers are arranged on the same horizontal level surrounding a container having a side wall for simplifying an image geometrical and signal transmission and echoing analysis based on a configuration of the same horizontal level. In one embodiment, the ultrasound transducers are distributed substantially around the sidewall for real time three dimensional (3D) image data acquisition. In one embodiment, baffle layer is shaped to reduce acoustic reverberation. The ultrasonic image scanning system can also include an image data processor for performing a three-dimensional volume and/or surface rendering for representing a scanned image of the organic object.

The ultrasonic image scanning can further include a computer aided diagnostic (CAD) system for processing a set of two-dimensional or three-dimensional image scan data for constructing and presenting tissue structural characteristic details of the organic object according to the set of image scan data. The ultrasonic image scanning system can further include a dense breast screen device to prescreen a dense breast as an organic object for carrying out an image scan by the ultrasonic image scanning system. In one embodiment, the ultrasound transducers are distributed substantially with a uniform circular arrangement for acquiring an ultrasonic image vector in a constant arc distance for reconstructing an image of the organic object. In one embodiment, the ultrasound transducers further comprising omni-directional high frequency ultrasound probes. In one embodiment, a scan controller is configured to automatically control an image scanning process and data acquisition by controlling the ultrasound transducers. The system can include a high density miniature connector to transmit signals between the scan controller and the ultrasound transducers. The high density miniature connector can include a high voltage semiconductor multiplex switch for controlling a real time signal routing.

In one embodiment, the invention relates to a method for ultrasonically scanning an organic object includes placing a plurality of modules comprising a plurality of ultrasonic transducers around a container, each module further comprising a high voltage multiplexer; and providing a baffle layer within the module in the areas between each of the plurality of ultrasonic transducers to reduce an acoustic reverberation using the baffle layer. The method can further include distributing the plurality of ultrasound transducers to substantially surround a circular probe. The method can further include distributing a plurality of ultrasound transducers substantially at symmetrical angular positions at approximately equal divisions of 360 degrees.

In one embodiment, the invention relates to an ultrasonic image scanning system for scanning an organic object includes plurality of modules, each module comprising a high voltage multiplexer and a plurality of ultrasonic transducers, the plurality of ultrasound transducers for transmitting the ultrasonic signal to the organic object through without asserting an image deforming pressure to the organic object, the modules distributed substantially around a two-dimensional perimeter of the container, the ultrasound transducers distributed substantially at symmetrical angular positions at approximately equal divisions of 360 degrees over the two-dimensional perimeter of the container; and a baffle layer positioned in the areas between each of the plurality of ultrasonic transducers to reduce an acoustic reverberation associated with the scanning the organic object. In one embodiment, the module includes a printed circuit board that supports each of the ultrasonic transducers and the high voltage multiplexer. In one embodiment, the baffle layer is shaped to reduce acoustic reverberation. In one embodiment, the ultrasound transducers are linear or phased array transducers. The organic object has an interior and an exterior and one or more tissue structures disposed in the interior. The exterior can include skin and other body object surfaces or surface structures. The interior can include fat, muscle, tumors, lesions, and other structures of interest. These structures can be imaged in three dimensions as can the interior or exterior of the object.

In one embodiment, the invention relates to ultrasonic image scanning system for an organic object having one or more tissue structures disposed therein. The system includes an immersible circular ultrasound probe having a circumference, the probe includes a plurality of modules arranged along the circumference, each module comprising a high voltage multiplexer, and a plurality of ultrasonic transducers, the plurality of modules defining a cavity configured to receive the organic object and a coupling medium, the plurality of ultrasonic transducers configured to scan the organic object in slices and obtain a three-dimensional image of the organic object that depicts one or more tissue structures disposed in the organic object; and a baffle layer positioned in each module in areas surrounding each of the plurality of ultrasonic transducers to reduce an acoustic reverberation associated with ultrasonic image scanning. In one embodiment, the ultrasonic image scanning system includes an image data processor for performing a three-dimensional volume and/or surface rendering for representing a scanned image of the organic object, the image data processor configured to render a volumetric configuration for the organic object using a tomography process. In one embodiment, the ultrasound transducers are distributed substantially with a uniform circular arrangement for acquiring an ultrasonic image vector in a constant arc distance for reconstructing an image of the organic object of a tumor or lesion disposed in the organic object.

In one embodiment, the invention relates to a method for ultrasonically scanning an organic object having one or more tissue structures disposed therein. The method can include placing a plurality of modules comprising a plurality of ultrasonic transducers around the organic object, each module further comprising a high voltage multiplexer; providing a baffle layer within the module to reduce an acoustic reverberation using the baffle layer; scanning the organic object in slices; obtaining a three-dimensional image of the organic object; and displaying one or more tissue structures disposed in the organic object.

In one embodiment, the invention relates to an ultrasonic image scanning system for scanning an organic object having one or more tissue structures disposed therein. The system can include a plurality of modules, each module comprising a high voltage multiplexer and a plurality of ultrasonic transducers, the plurality of ultrasound transducers for transmitting the ultrasonic signal to the organic object, the ultrasound transducers distributed substantially at symmetrical angular positions at approximately equal divisions of 360 degrees over the two-dimensional perimeter of the container, the plurality of modules defining a cavity configured to receive the organic object and a coupling medium, the plurality of ultrasonic transducers configured to scan the organic object in slices and obtain a three-dimensional image of the organic object that depicts one or more tissue structures disposed in the organic object; and a baffle layer positioned in the areas between each of the plurality of ultrasonic transducers to reduce an acoustic reverberation associated with the scanning the organic object.

These and other objects and advantages of the present invention will no doubt become obvious to those of ordinary skill in the art after having read the following detailed description of the preferred embodiment, which is illustrated in the various drawing figures.

### BRIEF DESCRIPTION OF FIGURES

The present invention is described in detail below with reference to the following figures.

FIGS. 1A and 1B are perspective views of a prior art scanning system disclosed in a prior art patent.

FIG. 2 is a side cross sectional diagram of an ultrasonic scanning system according to an illustrative embodiment of the invention.

FIG. 3 is a functional block diagram for showing the functions carried out by the ultrasonic image scanning system of FIG. 2.

FIG. 4 is a linear diagram for showing an arrangement of MLA (Multiplexed Linear Array) elements to channel bus connections according to an illustrative embodiment of the invention.

FIG. 5 is a linear diagram for showing an arrangement of MLA elements to channel bus connections according to an illustrative embodiment of the invention.

FIG. 6 is a top view of a module covered with baffle according to an illustrative embodiment of the invention.

FIG. 7 is a side cross sectional view of container implemented with a stepping motor for vertically moving a scanning sensor along screw rods according to an illustrative embodiment of the invention.

FIG. 8 is a top view of slices for showing a composite image of data acquisition during the scan process according to an illustrative embodiment of the invention.

FIG. 9 is a schematic diagram that illustrates a calibration pattern according to an illustrative embodiment of the invention.

FIG. 10 is a connection diagram that illustrates transducers connected to multiplexers for transmitting multiplexed scanning measurement signals to signal processing controller according to an illustrative embodiment of the invention.

FIG. 11 is a table showing an example of probe input to a high voltage multiplexer output connection implemented in a specific embodiment of the invention.

FIG. 12 is a side cross sectional diagram of an ultrasonic scanning system that includes a cylindrical transducer array according to an illustrative embodiment of the invention.

FIG. 13 is a functional block diagram showing the processes and steps the ultrasonic image scanning system of FIG. 12 is configured to perform according to an illustrative embodiment of the invention.

FIG. 14 is a schematic diagram of a two dimensional transducer array according to an illustrative embodiment of the invention.

FIG. 15 is a schematic diagram of an ultrasound probe that includes a two dimensional transducer array having a substantially cylindrical shape according to an illustrative embodiment of the invention.

FIG. 16A is a schematic diagram showing a baffle material disposed relative to a plurality of transducers according to an illustrative embodiment of the invention.

FIG. 16B is a schematic diagram showing a module with one active row of transducers producing parallel beams of acoustic waves according to an illustrative embodiment of the invention.

FIG. 17 is a schematic diagram of an ultrasound probe that includes a two dimensional transducer array having a substantially cylindrical shape disposed in a container relative to a body object according to an illustrative embodiment of the invention.

FIG. 18 is a top view of slices of a body object in a container obtained during a data acquisition scan according to an illustrative embodiment of the invention.

FIG. 19 illustrates a calibration pattern used with a two-dimensional transducer array-based probe according to an illustrative embodiment of the invention.

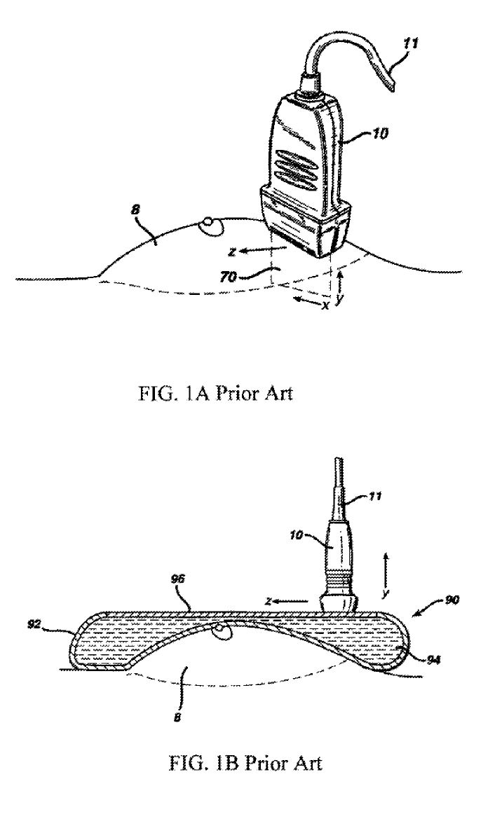

### DETAILED DESCRIPTION

In part, the invention relates to a three-dimensional ultrasound imaging system and related methods, devices, and subsystems suitable for scanning a body object such as a full breast (or partial breast) for image generation such as tomographic 3D imaging of the body object. The ultrasound imaging can be performed before using a probe that includes two dimensional circular or elliptical arrays of transducers. The probe is substantially cylindrical in shape and is configured for sliding in a container or is attached to a container sized to receive a body object of interest. In one embodiment, the ultrasound imaging is performed on an expedited basis relative to conventional scans such that the ultrasound scan of a body object, such as a breast, is performed at a time less than between about 1 second to about 10 seconds. In one embodiment, a body object, such as a breast, is scanned in less than about 5 seconds.

In one embodiment, a motor is not used to move transducers. Instead, a breast or body object is scanned by placing it in a container that includes a probe with transducers that are substantially stationary, but electronically addressable. In one embodiment, there are no moving parts in a given probe assembly other than the transducers which vibrate when sending or receiving acoustic waves. Selectively addressing rows of transducers that are disposed in a series of substantially parallel rows or substantially circular loops or bands allows an object of interest to be scanned from the bottom up or the top down. When the device is moving in motorized embodiments, it is possible for some blurring to occur as a result of the movement. In contrast, since there are no moving parts to cause the object under scanning to move or shift, the resultant 3D image remains substantially clear.

Prior to considering the cylindrical or other embodiments relating to a stationary transducer array discussed in more detail below, it is informative to consider some related features and embodiments of the invention and some known examples of imaging technology.

Medical imaging systems are known. For example, ultrasound imaging is discussed in the '080 patent and in U.S. Pat. No. 6,248,071, which are hereby incorporated by reference in their entirety for all purposes in this patent application.

The description and the drawings of the present document describe examples of embodiment(s) of the present invention and also describe some exemplary optional feature(s) and/or alternative embodiment(s). It will be understood that the embodiments described are for the purpose of illustration and are not intended to limit the invention specifically to those embodiments. Rather, the invention is intended to cover all that is included within the spirit and scope of the invention, including alternatives, variations, modifications, equivalents, and the like.

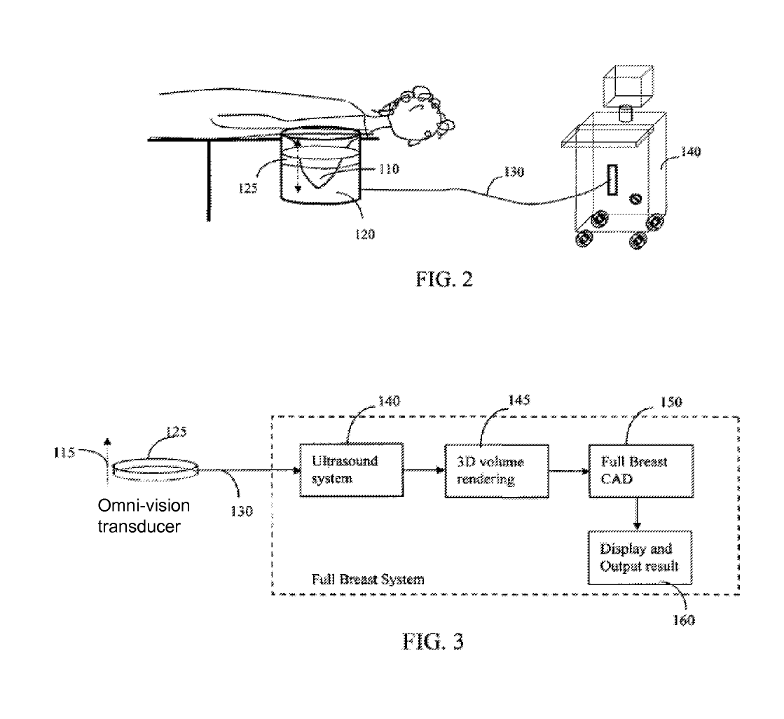

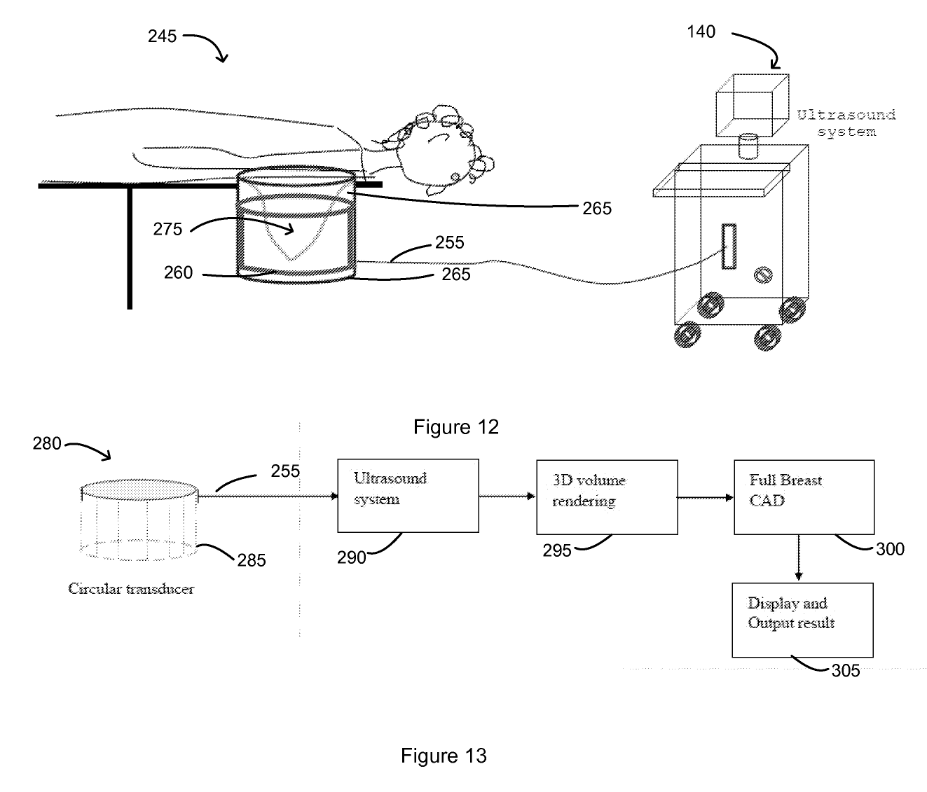

FIG. 2 is a schematic block diagram that shows a three-dimensional (3D) full breast scanning image acquisition system as an embodiment of the present invention. A breast 110 is within or at least substantially within a container 120. The container 120 is filled with liquid, preferably de-bubbled water. An ultrasound probe 125 is disposed in or around the container 120. The ultrasonic probe 125 is preferably an omni-directional high frequency ultrasound probe. In an exemplary embodiment as shown, the ultrasonic probe 125 is controllable to move in the container, for example, along a vertical direction of the container 120 in a linear up and down direction. The image scanning signals when collected by the probe 125 is then transmitted through a cable 130 to an ultrasound system 140 to carry out image display and data collection and analysis functions. As shown in FIG. 2, the ultrasonic probe 125 has a ring shape to move up and down in the container 120 that has a cylindrical shape. The ring shape can be extended to form a cylinder that remains stationary in some embodiments as described below in FIG. 12.

In an alternate embodiment, the interior of the container 120 is lined with a plurality of probe elements such as for example shown in FIG. 12. With a sufficient number of probe elements in 2D, physical movement of the probe is not necessary, and it is only necessary to switch on or address the appropriate probe elements, collect data, and then address probe elements at another position. The omni-directional probe, i.e., transducers, preferably is configured with sufficient probe elements, and/or sufficient 2D area coverage of the probe elements that the omni-directional probe can acquire the breast image in 360 degrees.

FIG. 3 shows the movement of the omni-directional transducer as the ultrasonic probe 125 moves along a vertical direction 115. The data of scanning image is transmitted through the cable 130 to the ultrasound system 140. The ultrasound system 140 first carries out a 2D composite image construction. The next step is to perform the 3D volume rendering process 145 of the three-dimensional image signals. Next, a full breast CAD process 150 is performed to construct a data array for representing the three dimensional image data. The image scanning results are displayed for diagnosis and as output data for further processes and analyses (160).

In contrast to the conventional freehand and water-bag acoustic scanning approaches, the system as shown above provides an acoustic probe to scan a subject body part from different positions without varying the pressure applied to the subject body part. Furthermore, the probe is now enabled to simultaneously scan the subject body part from non-parallel directions without varying the pressure applied to the subject body part.

The water in the container 120 serves the function as an acoustic coupling between the scanning surface and the breast. The water is preferably de-bubbled water. The water is automatically filled and drained from the container via conduits (not shown). The container is kept hygienic with regular disinfection, and the container may be configured to automatically dispense disinfection agents via a conduit. In one embodiment, the movement of the probe is linear only, without rotation, for the sake of simplicity and reliability.

In one embodiment, multiple containers and probes are provided to accommodate different sizes of breasts. Preferably, a wire target of known configuration is provided to the end user to place within the container to provide a test pattern so that the system can calibrate itself for the scan converter algorithm. In one embodiment, there is a baffle around each transducer to absorb energy, which may cause acoustic reverberation. The system may be configured to have, for example: 40 mm probe FOV (Field Of View) and 7.5 MHz or higher 128 or 64 elements, 12 to 20 modules per circumference of probe, 20 cm diameter maximum for the acquisition window, 30 cm height, and various other features and parameters as described herein. Depending on design choice and depending on application other elements or configuration of similar designs may be also be implemented. The calibration is for the scan converter (R-Theta to Rectangular) algorithm to map all the images from different angles into a circular image. The above numbers represent exemplary implementation of the transducer for each multiplexed module.

Preferably, a scan controller implemented in a computer, e.g., the ultrasound system 140 that includes a scan controller, automatically controls the image scan and data acquisition processes. By properly setting up the control processes, standard types and amount of signals and data are generated and acquired through the image scanning processes. The results of image scanning would then not depend on the level of skill of an image scanner operator and human errors can also be minimized. The speed and cost of carrying out such scanning process is also significantly reduced while more reliable and accurate diagnosis can be provided by consistently using standardized scanning configurations and data analysis processes carried out by highly accurate CAD and image processing systems for detail review by radiologist. The 3D volume image provides a consistent basis for correlation and comparison over a period of time to track whether there are changes in the scanned image over time.

Referring to FIGS. 2 and 3 again, the ultrasound system 140 may be implemented as a sonogram system that includes a computer-controlled acoustic probe 125. The acoustic probe 125 scans the subject body part from different positions without varying the pressure applied to the subject body part and preferably from different non-parallel directions without physically touching the subject body part. The de-bubbled water serves the function as coupling medium for transmitting acoustic waves for receiving a scanning image signals back from the subject body part. The scanning image signals are then received and stored based on the positions of the probes and the timing sequence of these signals for rendering a volumetric configuration for the scanned body part by applying appropriate tomography techniques. In one embodiment, the transducers and other data collecting element directly contact the subject body part during the ultrasound data collection session. This contact can be periodic or continuous throughout the scan.

For actual implementations, the ultrasonic probes may utilize probe technology with fine AWG cable. An high voltage 20220 or Hitachi 3290 chip or the like may be used. A card-bus connector (68 pin) or the like may be used. Reverb cancellation is preferably included according to any competent technique, for example, any competent conventional reverb cancellation technique. The card-bus connector provides the multi-wire connection in between the probe module. After many single angle probe modules are linked together, it forms a complete 2D circulator probe. The final outputs are brought to the ultrasound system 140 through fine gauge wire cable (e.g. AWG 36). The high voltage 20220 chip is a high voltage semiconductor multiplex switch. It is used in the single probe module to provide the real time signal routing. When the acoustic wave travels in the water, it may bounce back and forth in the media. The baffle serves the purpose of muting this secondary reflection artifact. The controller may also include a CAD capability for processing three-dimensional data to obtain accurate compound tomography data suitable for generating tomographic images.

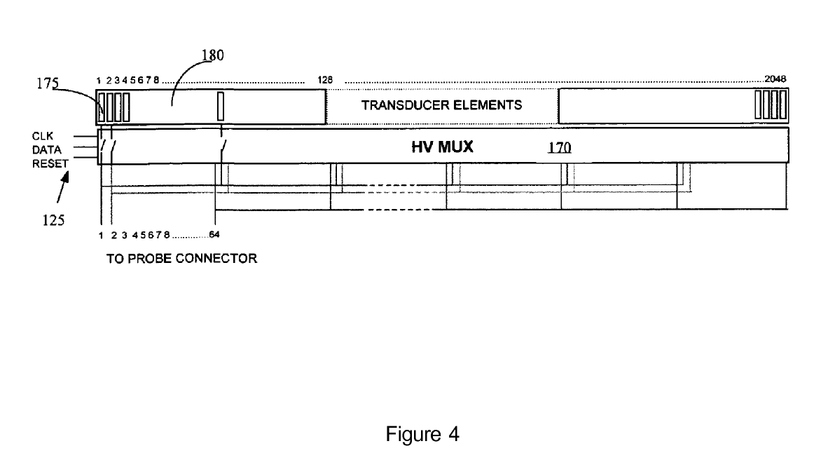

Referring to FIG. 4 for the configuration of the ultrasonic probes implemented with omni-directional transducer arranged according to the MLA configuration to multiplex the element (>2048) bus to the channel (64) bus. Omni-directional allows the image to be acquired at 360 degree in real time. The MLA provides the signal routing from each individual transducer module to the ultrasound system.

In a specific embodiment, there are 2048 elements arranged in a circle, and the high voltage multiplexer 170 is configured in a way that the output from every sixty-four transducer elements 175 are multiplexed together. In each individual probe module, there is an array of transducers with High Voltage Multiplexer. The array further consists of 64-128 elements of piezo-transducer in one substrate in order to form a focus beam electronically. Each multiplexer (MUX) 170 has a separate control bit to turn the high voltage switch on/off. In the beginning after the system reset all these high voltage switches, logic high will be shifted into the high voltage multiplexer control register to connect the selected element to the output until all 64 elements are turned on, thereafter, it will slide the element until it reaches the end of the first group of 128 elements. Then the transducer element 175 switches to the next group and repeat the same procedures. A similar scheme for controlling the 2D probes described below such as shown in FIGS. 12, 14 and 17 for example.

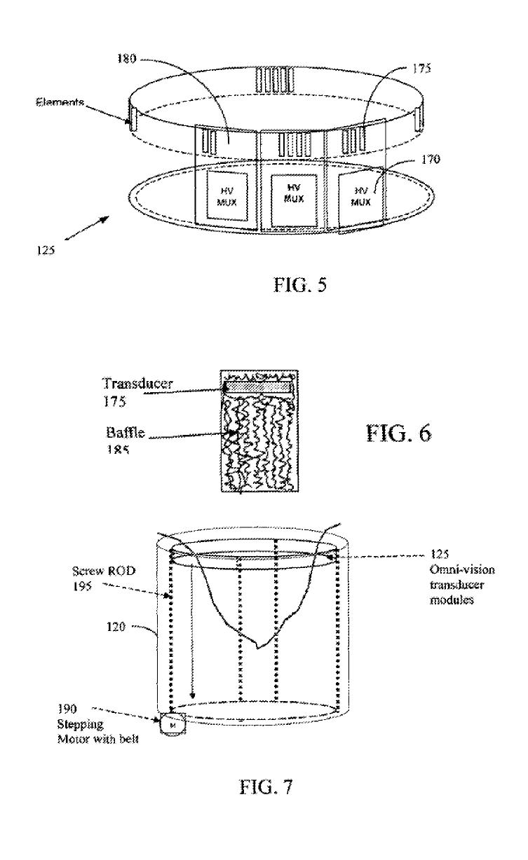

Referring to FIG. 5, each group of transducers supported on a printed circuit board module 180 has a width of 40 mm. The circumference is sixty-four centimeters, i.e., about twenty centimeter diameters, to accommodate sixteen transducer groups supported on the printed circuit board 180. Each printed circuit board module 180 has one hundred and twenty-eight (128) element transducers 175 and high voltage multiplexer 170. Inter-module connection can be a flex cable with miniature high-density (>68) card bus connector, or a circular printed circuit board at the bottom, or even a direct connection in between printed circuit board. The ultrasound system handles the omni-directional circular probe as a circular MLA with 2048 elements in sixteen (16) groups.

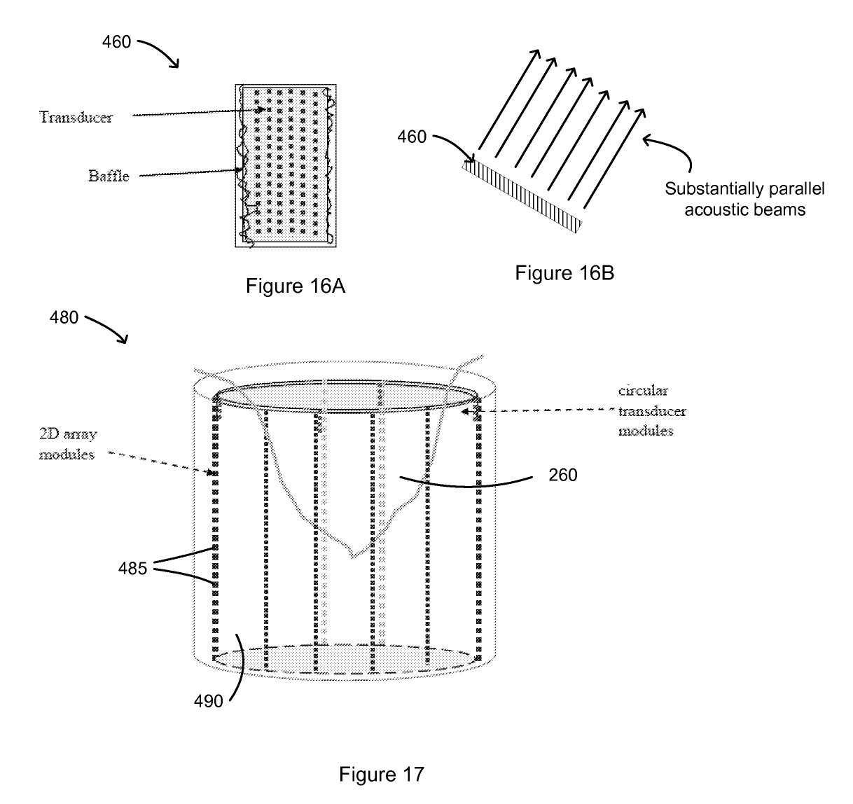

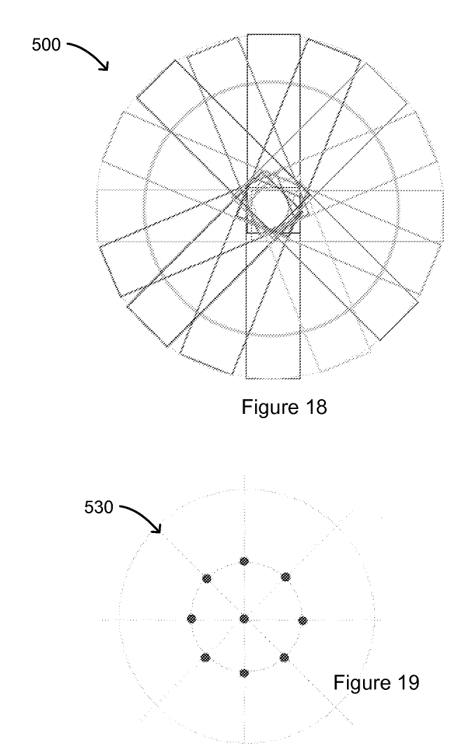

Instead of the MLA configuration as described above, an alternate embodiment is to place the 2048 probe elements in a uniform circular arrangement, instead of 16 modules at 16 different angles, for acquiring each image vector in a constant arc distance and reconstruct the image. The acoustic energy probably would be concentrated around the center location and may cause the energy intensity to exceed the governmental regulatory limits, e.g., FDA limit. The image density vectors may not uniformly distributed with higher density distributed in the center and much lower density distribute on the edges and that may affect the image quality due to these non-uniform distributions of image density vectors. Additional details relating to another embodiment of the invention that addresses this issue is described below with respect FIGS. 18 and 16B. The new embodiment generates parallel beams in each module, and these beams overlap from module to module to cover the whole area. As shown in FIG. 18 and FIG. 16B not all beams are focusing at the center point.

A further limitation for such configuration is that it cannot compound the image with different speckle distribution since the speckle changes when the image vector is acquired from different angle. However, since this embodiment has the advantage that the configuration appears simple and easy to implement conceptually, it is also included as one of exemplary embodiments in this invention as well. In one embodiment, in one area inside the scanned body, multiple beams at different angles are acquired and these beams are compounded together to reduce the speckle or acoustic shadow.

Another configuration for implementing this invention is to apply one module, or several modules as ultrasonic probes for transmitting ultrasound waves to the scanned body object for receiving the feedback signals to construct an image of an object being scanned. In one embodiment, the motor is applied to drive the probe module or modules to circularly move around the container to acquire the image by collecting data from an image scan with the ultrasound probes placed around three hundred and sixty degrees. Such operation may require longer period of time than the distributed scanning probe configuration as described above. Another concern of high-speed motion of the ultrasound probes in the container is the generation of bubbles that may affect the accuracy of the image scanning. The circular move around the peripheral edge of the container also leads to more complicate cable arrangements. Furthermore, alignment and angular calibration operations of the scanning probes may be necessary when multiple shots in different angles are likely to produce overlapping and compound images.

FIG. 6 shows the areas 185 on a printed circuit board other than the transducer 175. For the purpose of absorbing the acoustic energy to prevent reverb echo, the areas between the transducers are covered with rubber baffle formed with an irregular shape or highly attenuating materials.

FIG. 7 illustrates that the whole transducer 125 can be moved up and down with a stepping motor 190 supporting the movements with screw rods 195. There is preferably no rotation involved. The movements of the transducer 125 can be controlled by a controller included in the ultrasound system 140 implemented with a software that sets a maximum displacement. The maximum displacement may also be based on the length of the subject body image for scanning. When there is no echo according to the reflected signals from the whole image, it is determined that the probe 125 has moved to the end of the tissue volume 110. The control of the stage motor 190 is through the RS232 or USB port or other similar or equivalent connecting ports from a host personal computer that may be implemented in the ultrasound system 140.

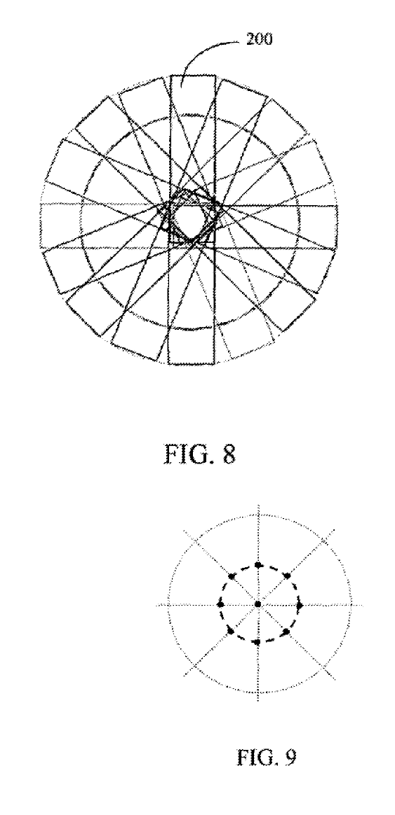

FIG. 8 shows divisions or slices 200 of the image data acquired from the transducers distributed over a circumference covering multiple rectangular areas. As shown, each set of transducers are receiving signals from a different angle. With the acquisition of the same image area from different angles, a composite image is formed through tomography using image processing techniques similar to MRI image processing. With the motor movement, a volume image can be acquired for the 3D viewing. For a 20 cm diameter image slice, each segment image only needs to process of a length of 10 cm. An array probe can be utilized that is competent for such penetration; for example, a 7.5 MHz linear array transducer module may be used. For the image acquired from each module, the system may perform a scan conversion with different angles and compound the images together.

Referring to FIG. 9, for the purpose of assuring the images are aligned properly, there is a wire pattern phantom used to calibrate during the image installation. A phantom is any dummy target used to simulate the echo reflected from scanned object according to transmission of ultrasound waves. The wire targets are in a fixture for calibration as described above. The purpose of such calibration is mainly for adjusting the geometric error due to the assembly of the 16 probe modules when operated together during the process of a scan conversion.

A CAD system and methods are incorporated that characterize the features of the disease and make a best estimation to guide a radiologist's review of the scan. A 3D volume rendering display can be shown on the screen to help the doctor make a diagnosis. The CAD process usually starts from volume rendering the 3D data sets of image, and display the 3D image on the screen. The user can rotate the image in any angle, set the different opacities of the object to identify the suspicious lesion. An advanced CAD algorithm can analyze the image with the cancer features, for instance, the smoothness of the lesion boarder, the shadow behind the lesion, etc. This type of information can help, not to replace, the radiologist in making the decision. The surgeon can also have an idea about the size and location of a tumor before the operation, and the oncologist can trace the tumor size during the treatment using a 3D image generated from the probe data.

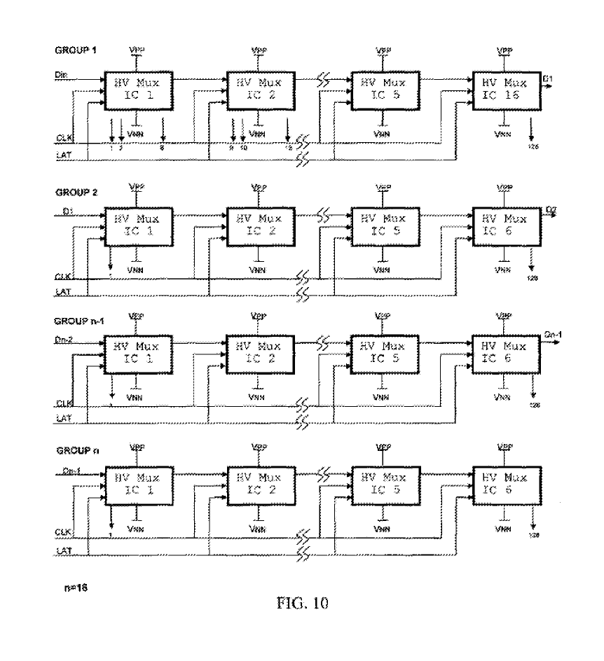

Referring to FIG. 10 for an implementation of the omni-directional probe high voltage multiplexer connection. In one embodiment, there are 2048 transducer elements of Omni-directional probe multiplexed such that 64 (or less) consecutive elements are allowed for selection at any one time. Each time an aperture of image scanning is decided, control data is clocked into the shift register of the high voltage Mux Ics (Hitachi high voltage mux3290), after transferring 64 bits ‘1’, the system will start to clock in ‘0’ for each line increment until it reaches to the end of the group, and the setting moves to the next group. At the output of the high voltage Mux, module 1 to 16, element number 1 and 65 are connected together and become CH0, that is equal to element 1. Likewise, module 1 to 16, element number 2 and 66 are connected together and become CH1 and so forth.

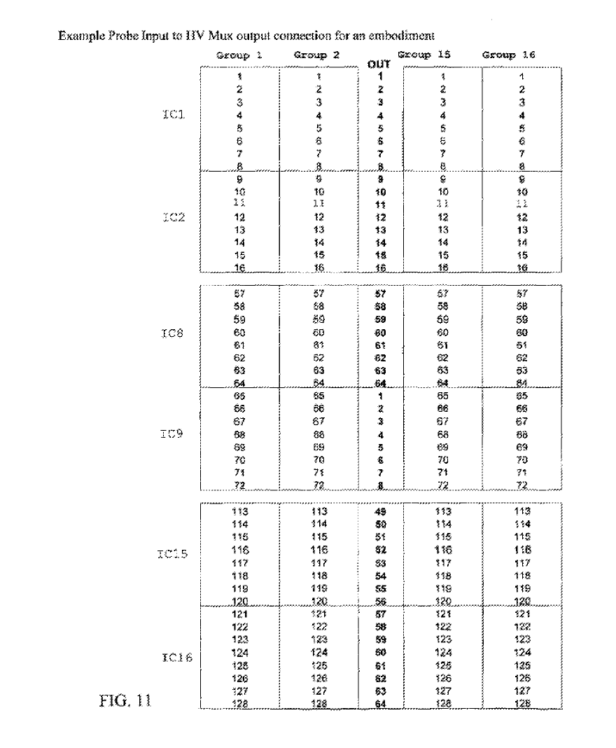

An example Probe Input to high voltage Mux output connection for an embodiment is shown in FIG. 11. The arrangement of this connection allows the invented probe device to be compatible with the conventional ultrasound system therefore, the scan conversion algorithm and the hardware platform can be adapted for this purpose.

### Stationary Two-Dimensional Transducer Array Embodiments

The medical device industry has been long searching for a solution of detecting breast cancer at an early stage. The mammogram was chosen for breast screening according to the healthcare policy mainly because of the cost efficiency of use. However, the accuracy of mammograms is still questionable for women with dense breast tissue. Ultrasound scans can complement mammograms, but variations in a technician's skill needed to perform the scan, the extra cost, and the extra time associated with such scans have been an obstacle to their widespread adoption.

Although useful for many applications, a probe having a ring or loop geometry that includes a 1 dimensional (1D) transducer band driven by motor configured to acquire data for a three-dimensional (3D) volume has certain limits with respect to the speed of data acquisition. The time needed to translate the probe over a distance using a motor or mechanical system imposes additional time constraints. Faster motors and other techniques can be used to compensate. However, using a probe that is substantially stationary with a two-dimensional transducer array is also an option as described herein.

Embodiments of the invention address some of these issues and others. In one embodiment, the ultrasound probe includes a plurality of transducers that are fabricated using a Lead zirconate titanate (PZT) material. In one embodiment, a suitable PZT semiconductor material is used in conjunction with one or more microelectromechanical systems or devices (MEMS) to form a two-dimensional circular array of PZT semiconductor transducers. In one embodiment, MEMS such as for example the devices described in, for example, U.S. Pat. No. 7,420,317, which describes MEMS that include piezoelectric actuators, can be used. The role of the MEMS is to implement the highly dense transducer elements with the semiconductor process technology, and the chance of combining the circuitry (e.g. HV MUX, preamplifier, pulser) together in the same substrate to reduce the wiring. The MEMs are used as the active wave producing and receiving elements in one embodiment of the transducer array, and the associate circuitry can be for signal preprocessing or controlling. This can speed up the 3D volume acquisition by at least 10 fold relative a motor driven embodiment and allow the transducer to touch the breast if necessary. The speed increase in terms of the scan time arises because the beam steering is controlled electronically using the HV MUX logic, which operates in the microsecond range.

In one embodiment, using electronic devices or circuits to perform beam steering or transducer addressing, without a motor, speeds up the scan time. By using a MEMs, this enables the circuitry to be integrated in the same substrate, which reduces the required wiring. Even if the transducer touches the breast, it is still consider as stationary during the signal acquisition. In the non-stationary embodiments, the moving motor can cause the image to blur during data acquisition. However, if the acquired images are used for further comparison in the later time, it is better to not deform or change the shape (by touching) of the breast too much because the CAD may then have difficulty in correlating the images. Together with a 3D Computer Aided Diagnostic (CAD) software algorithm, breast cancer can be detected early during a screening session that uses the ultrasound data obtained with the 2D circular array. Lesions and tumors can be resolved within the breast using a given cylindrical probe embodiment. This offers particular advantages for diagnosis of cancer when a patient has dense breast tissue. In one embodiment, a technician performs the scan and CAD and reviews or a radiologist reviews the resultant data and/or images.

As discussed above, the invention relates to full breast (or partial breast) three-dimensional ultrasound imaging. The ultrasound imaging can be performed using a two dimensional circular or elliptical array of transducers. That is, the transducers can be disposed over a two dimensional surface having a circular or elliptical cross-section. In one embodiment, the ultrasound imaging is performed faster relative to conventional scans such that the ultrasound scan of a body object, such as a breast, is performed at a time less than between about 1 second to about 10 seconds. In one embodiment, a body object, such as a breast, is scanned in less than about 5 seconds. In one embodiment, the through-put rate for scanning two breasts is about 1 minute. The throughput rate refers to a scan performed relative to a 15 cm diameter and 15 cm depth scan area. Additional details relating to ultrasound probe and system embodiments in which one or more transducer element remain substantially fixed are described below with respect to FIGS. 12-19.

FIG. 12 is a schematic block diagram that shows a three-dimensional (3D) full breast scanning image acquisition system 245 as an embodiment of the present invention. An ultrasound subsystem 140 is shown in electrical communication with a cable 255. In turn, the cable 255 is in electrical communication with an ultrasound probe 260. As shown, the probe 260 has a cylindrical shape and is disposed in a container 265. The probe can be a circular, elliptical, polygonal, or have another type of cross-section. A body object shown as a breast 275 is within or at least substantially within the container 265. The container 265 is filled with liquid, preferably de-bubbled water.

In one embodiment, the ultrasound probe 260 is disposed in the container 265. The probe 260 can slide into the container or be affixed thereto. Thus, the probe 260 and the other probes described herein are submersible or immersible in a fluid such as the fluid used to fill the container 265. The ultrasonic probe 260 has a cylindrical shape defined by a substrate or support. The substrate is formed such that the probe 260 is open at one or both ends such that a body object 275 can be received by the probe 260 in the cylindrical inner cavity defined by the substrate. The substrate can include a composite material, PZT, ceramic, crystalline materials, capacitive materials, semiconductor materials, and other suitable transducer or piezoelectric materials. As shown, a planar substrate or support can be rolled or folded to form the probe 260. Rectangular panels can also be used to form the probe 260.

In an exemplary embodiment as shown, the ultrasonic probe 260 is stationary. Instead of moving the probe, a plurality of transducers are disposed along the surface of a substrate, support or backing layer which is formed to have a cylindrical shape. The image scanning signals, when collected by the probe 260, are then transmitted through the cable 255 to an ultrasound system 140 to carry out image display, tomography, data collection, CAD, tomography, rendering and/or analysis functions.

In one embodiment, the probe 260 is a circular or cylinder type probe that includes one or more high frequency ultrasound transducer modules disposed around the inner wall of the container 263. These transducers are configured to acquire the 3D breast volume without moving the probe. In one embodiment, a given transducer module can include one or more microelectromechanical systems (MEMS). The probe 260 can include a plurality of panels (or modules) that each connect to another panel (or module) along an edge such that panels define a two-dimensional closed surface having a substantially cylindrical shape. The cross-section of the probe 260 can include various suitable shapes, including without limitation, a substantially circular cross-section, a polygonal cross-section, and a regular polygonal cross-section. In one embodiment, the number of sides of the polygonal cross-section is determined by the number of panels connected together to define the closed surface which faces the body object. Concave or convex curved transducer arrays may also be used. The control unit steers the beam to regulate the transmit energy such that it does not concentrate at one spot. The use of parallel or substantially parallel beams that cover different planes or lines of the object, and not just the center, also help reduce heating. Further, the scan conversion process that turns data into one or more images is configured to cover a fan shaped beam in one embodiment.

With a sufficiently large number of 2D probe elements such as transducers, physical movement of the probe is not necessary, and it is only necessary to trigger the appropriate probe elements, such as individual transducers arranged to form a two-dimensional array on or within the surface of the probe 265. Thus, by selectively addressing probe elements, a scan of a body object 275 can be performed without moving the transducer. The omni-directional probe, preferably is configured with sufficient transducers, and/or sufficient mobility of the probe elements that the omni-directional probe can acquire the body object 275 image in 360 degrees. The number of transducers can range from 2048 to 65536 for example. The ultrasound system normally has 64 to 128 channels for the beam forming. Given the thousands of transducer elements that are providing data for 64-128 channels, a multiplexing scheme is used. For an embodiment of a system having multi-thousand channels, the HV mux and can transmit or receive all elements simultaneously.

In contrast, to the motor drive probe of FIG. 3, FIG. 13 shows an alternate embodiment of an ultrasound system 280 that uses a cylindrical probe 285 that is stationary during a scan. The system 280 of FIG. 13 illustrates an ultrasonic probe 285 suitable for insertion into a container or for defining or otherwise being attached to a suitable body object and fluid receiving container. Once the probe 285 scans the body object, the data obtained to generate an image is transmitted through the cable 255 to the ultrasound system 290. The ultrasound system 290 triggers the probe 285 to send and receive acoustic waves. The wave propagation and reflection from the object are captured as electrical signals which are transformed and rendered to include interior views of the body object. This process can be performed during the 3D volume rendering process 295 of the three-dimensional image signals. Next, a full breast CAD process 300 is performed to construct a data array for representing the three dimensional image data. The image scanning results are displayed for diagnosis and as output data for further processes and analyses 305. Lesions and tumors can be displayed with different color or hatching.

With respect to FIG. 12, the container 265 typically includes a coupling fluid or gel to transmit and receive acoustic waves into and from, respectively, the body object 275. In one embodiment, this coupling fluid is water. The water in the container 265 provides an acoustic coupling between the scanning surface of the probe 285 and the breast. The water is preferably de-bubbled water. In one embodiment, the water is automatically filled and drained from the container via conduits (not shown). The container is kept hygienic with regular disinfection, and the container can be configured to automatically dispense disinfection agents via a conduit.

In one embodiment, multiple containers and 2D dimensional cylindrical probes are provided to accommodate different sizes of breasts. Thus, the container 265 and the probes 260, 280 can have heights and diameters that vary over different embodiments to accommodate different body objects and different sizes of particular body objects such as breasts.

In one embodiment, there is a baffle around each transducer to absorb energy, which may cause acoustic reverberation. The ultrasound probe and the other subsystems can be configured with respect to various parameters and design features. For example, in one embodiment, the field of view (FOV) of the 2D transducer-based probe is 40 mm×120 mm. In turn, the frequency of the probe can range from about 7.5 MHz to about 10 MHz or higher. In one embodiment, the transducers used in the probe remain substantially stationary. In one embodiment, the height of the probe ranges from about 10 to about 20 cm. In turn, the circumference of the probe can range from about 30 cm to about 45 cm. In one embodiment, the probe can have any suitable open or closed surface geometry having active transducer elements disposed in or on the surface such that the elements form an array that generates acoustic waves that are arranged or propagated in a substantially parallel configuration.

In one embodiment, the two dimensional probe is formed from a plurality of substantially flat or planar modules. Each module can include an array of transducer elements that form a J×K array of N transducers. The array can include various numbers of rows and columns of transducer elements. In one embodiment, J is equal to 128 and K is equal to 384 such that the probe includes 128×384 transducer elements per module with N equal to 49152. Thus, if there are between 8 and 12 modules, the number of transducer elements can range from about 393,216 to about 589,824. In turn, around the circumference of the probe, in one embodiment, the number of modules that include a plurality of transducer elements can range from about 8 to about 12 modules. In one embodiment, the scan performed using a two-dimensional cylindrical probe is about 20 frames per scan. The total time for a given scan can be about 5 seconds per body object. 1 mm in z for 120 mm refers to the 6dB beam width of the conventional 1D probe image. In one embodiment, this same level of resolution is used in the z axis scan direction to prevent missing a significant lesion or other tissue structure of interest.

In one embodiment, the image depth per volume is 120 mm per frame. The 120 mm refers specifically how far into the body object is data collected per volume from chest to nipple. The probe can be configured to scan objects of varying height and thickness. In one embodiment, the probe is configured to scan objects between 0 to about 15 cm in height. Depending on design choice, body object of interest, or the ultrasound application, other elements or configuration of similar designs may be also be implemented.

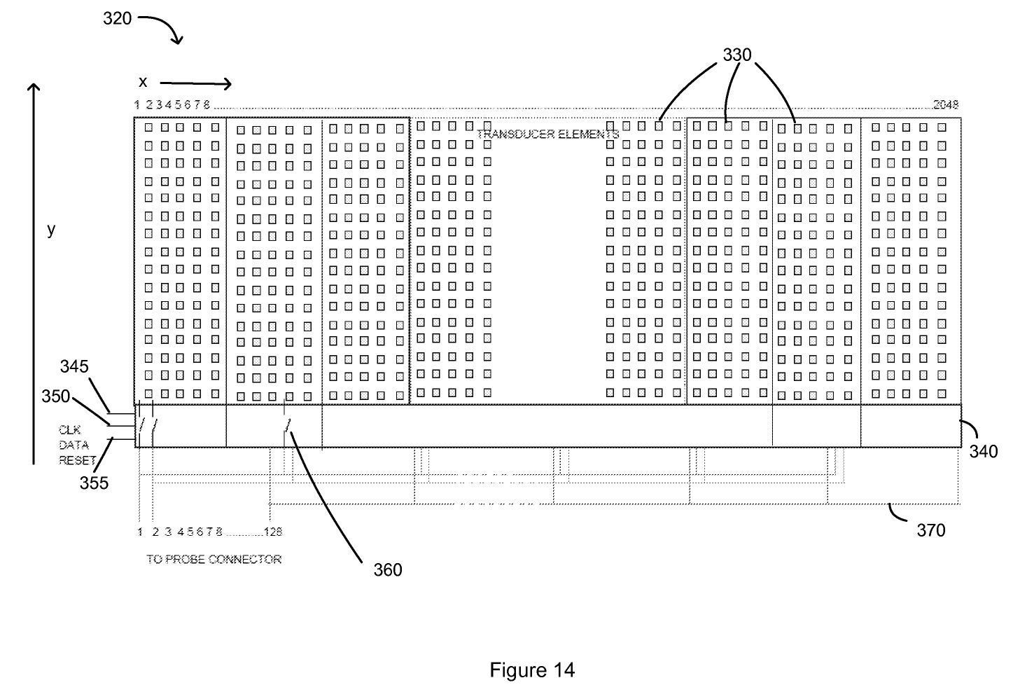

FIG. 14 shows an ultrasound probe 320 in an unrolled flat configuration for the purpose of depicting the transducer array. The probe 320 includes a two dimensional flat transducer that includes an array of transducers. In one embodiment, each transducer 330 includes a piezoelectric material such as PZT. Accordingly, in part, FIG. 14 shows a two-dimensional probe configured to multiplex data from the transducers 330. A horizontal axis is shown as an x-axis having an arrow that points to the right and a vertical axis is shown as y-axis having an arrow that points up.

Piezoelectric materials such as certain ceramic, crystalline materials, capacitive micromachined ultrasonic transducers (CMUTs), other micromachined ultrasound transducers (See for example U.S. Pat. No. 7,420,317 for other transducer materials and configurations suitable for use with the embodiments described herein), PZT and micromachined electromechanical (MEM) transducers are suitable for use in a transducer array. Specifically, such piezoelectric materials can be cut to form a two row multicolumn array, although any suitable n by m array is possible. In one embodiment, a unitary block or piece of piezoelectric material can be cut to form a transducer array or a component row of a transducer array having kerfs filled with a boundary material to define active elements. A kerf refers to a cut, incision, or groove.

In one embodiment, electrodes are attached to the piezoelectric elements that make up the array such that each element is electrically addressable using an applied voltage. The kerfs formed in the array can be filled with a suitable lossy material, such as a rubber or a polymer, and the piezoelectric components can be attached to a suitable backing material. Thus, an array of electrodes in a given module can focus an acoustic beam suitable for generating a three-dimensional image of the exterior and interior of a body object.

In one embodiment, the cylinder-shaped transducer array or arrangement uses one or more circuit elements or devices 340. The elements or devices 340 can include a high voltage switch or multiplexer 340 to multiplex the multi-signal transducer element (>128×384×12) bus to the 128 line signal bus. The switch or multiplexer can be implemented using a semiconductor device or a MEMs in one embodiment. Given that 128×384×12 elements (128×384 elements in 12 modules) are arranged in a circular cylinder, the MEMS and high voltage multiplexer is configured as one line below. Clock (CLK) 345, data (DATA) 350, and reset (RESET) 355 lines are shown.

Specifically, the probe 320 is configured such that there are 128×384 elements from each module that are linked together through a high voltage multiplexer or switch. Each multiplexer has one control bit or signal to turn the high voltage switch on/off. An exemplary high voltage switch 360 is shown. In the beginning, after the system resets all these high voltage switches to an off state, a logic high signal will turn on the high voltage multiplexer so the 128×384 elements can connect to the global bus. At the end of each sub-frame, the multiplexer enables signal transition to the next module for sub-frame image acquisition until it cycles or otherwise collects data from all 12 multiplexer modules that encircle the breast for one slice of imaging data. Each slice can be used to tomographically render a three-dimensional image of the breast. The transducers 410 are in electronic communication with one or more MEMS. In one embodiment, the MEMs are used to provide the active transducer elements in the array using PZT, CMUT, or another material suitable for use in a semiconductor-based embodiment. This is in contrast with die sawing of ceramic PZT, for example.

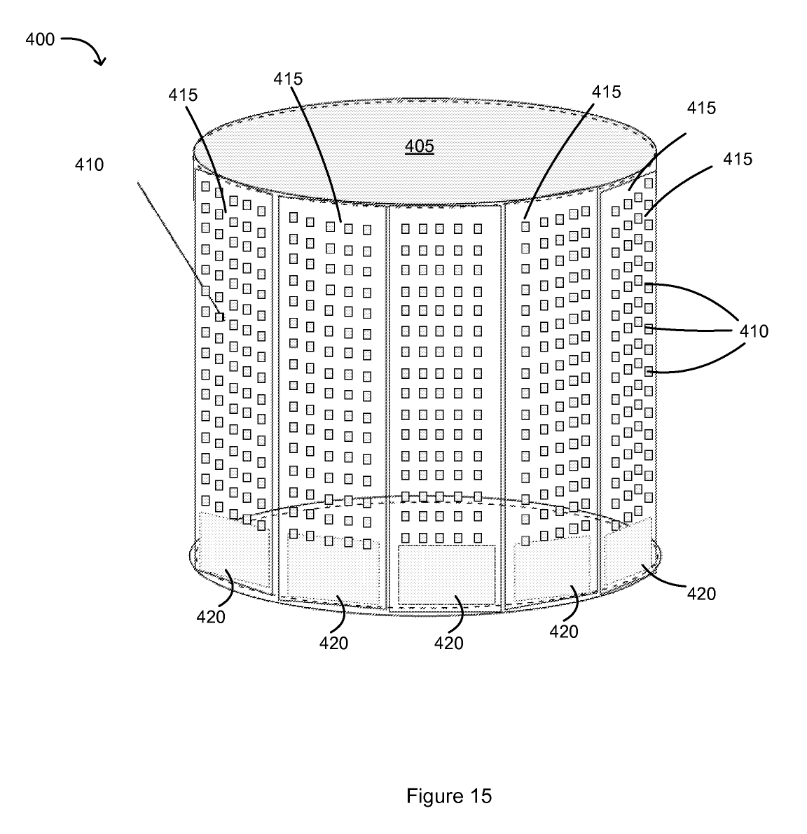

FIG. 15 shows an ultrasound imaging probe 400 that includes a plurality of transducers 410 arranged along a two-dimensional surface 415. The probe shown may have different diameter transducer module to fit different breast sizes (A-E size, etc.). Thus, in one embodiment, different containers can be used or a larger container can be used with the probe suspended therein and supported relative to the sides and bottom of the container such that different probes can be used.

During a scan or ultrasound data collection session, the set of data obtained with respect to a given body object is over determined or redundant in one embodiment. For example, overlapping scans from each opposing side of the probe yield extra data. Thus, the probe embodiments can perform compound ultrasonic imaging from different angles. Specifically, an image or ultrasound data set obtained with respect to the same area of a body object, such as breast, can be acquired several times from different angles and compounded to reduce the speckle and improve image quality.

In one embodiment, the speckle is a type of coherence interference due to the multiple energy sources. Thus, the speckle can occur as a result of constructive and destructive interference on the focal area. If the angle of the acoustic wave source changes in a given device, the speckle pattern changes. By correlating or adding these images (with different speckle) together, the image quality improves. Because the breast is relative stationary (comparing with heart), temporal averaging will improve the noise. Otherwise, for a moving object, this type of averaging can blur the resultant image.

Spatial compounding of image data can also be performed such that data associated with different angles and sections of a body object can be mixed and combine to generate a tomographic image or data set for the body object. In addition, the system can also perform 3D data ray casting for ‘see through’ breast imaging at different angles. During 3D data processing, when the viewing angle changes, the body object is rendered in real time to show different lighting, gradient, and raycasting features by using different pixel elements. If the data processing steps use or trigger on the maximum data, it will show the bone structure of the patient being scanned; and if data processing steps use or trigger on the minimum data (dark), it may show the blood vessel or cyst. Therefore, by configuring the data processing system to use the average data, the system is better configured to display a tissue structure of interest such as lesion structure or mass.

As shown in FIG. 15, each transducer has a width. In one embodiment, each transducer has a 40 mm transducer width. Therefore, the total number of modules required for a 48 cm circumference (15 cm diameter) probe is 480 mm/40 mm which equals 12. Each printed circuit board module has 128×384 element transducer module. Each module can also include an electronic device or a circuit element 420 as shown. The elements or devices 420 can include a high voltage switch, multiplexer, control element, MEMS, or other suitable device to send, receive, and process signals being sent to and received from the probe. Inter-module connection can be made with a flex cable and a miniature high density (>84×2, or 140 pin) connector, or a circular printed circuit board at the bottom of the probe, or a direct connection in between the adjacent printed circuit boards. The ultrasound system such as system 140 is configured to control data collection, clocking and signal processing to process the circular probe 400 as a regular 128 transducer element flat probe and process the data associated with the number of modules used in the probe 400.

In one embodiment, a single pin or line or multiple pins or lines from the control and/or imaging system triggers the frame pulse. The device uses this signal to clock a counter/demultiplexer for selecting the next high voltage multiplexer in a sequence. In one embodiment, the signal lines from the system are configured for use with the 128 active elements or transducers in each row of a module or panel. A power supply having an isolation transformer can be used to drive the probe in response to a control system. A negative high voltage supply (Vnn), a positive high voltage supply line (Vpp), and logic power supply line such as +5V line are in electrical communication with each module and originate from the power supply. In one embodiment, the pulse line (sub-frame sync), Reset line, control signals are connected to the system through RS232 or USB interface. The probe has a probe ID for the system 140 to generate the scan sequence.

In one embodiment, PZT semiconductor probe technology with a MEMS is used to reduce the number of probe outputs 128 wires to the system 140. In one embodiment, this reduction in outputs is accomplished by using an integrated high voltage multiplexer or pre-amplification/pulser circuits. The 2D cylinder transducer arrangement with semiconductor or ceramic PZT allows the transducer to touch the breast and with much faster scan time and accuracy.

The cylindrical probe embodiment described herein such as those depicted, for example, in FIGS. 12, 13, 14, 15 and 17 can be configured such that the acoustic waves directed towards a given body object are controlled to reduce excessive heating. Preferably, a body object such as a breast should remain within a pre-determined temperature range during a data collection event. In one embodiment, an ultrasound probe is configured such that the acoustic energy will spread around the whole breast area rather than at one spot. In one embodiment, the beams incident acoustic beams from each module are in parallel and not all focusing at the same spot. Thus, each transducer element in a given row is directing a beam in front of it, many of which are offset with respect to the sample. An example of this is shown in FIG. 16B. This spreads the acoustic waves over the length of each modules such that they are not all directed to a common center spot. This also reduces undesirable tissue heating. Further, the average temperature of the breast varies between about 37 and about 40 during a scan which is within the FDA limits of 43 degrees Celsius.

The probe is configured such that it scans the whole breast and performed imaging in 3D volume without compressing and deforming the breast soft tissue. This is particularly useful for dense breast tissue and improves image quality and a better signal to noise ratio results. In one embodiment, the probe is configured such that the surface of the probe can contact the breast during an ultrasound scan without losing image quality. The cylindrical probe embodiments can also be configured as a waist belt for multi-angle abdominal organ scanning. The shape can be any concave arc curve which can be used to scan a liver, a kidney, a baby, a leg scan or another body part. The shape does not need to be circular.

FIG. 16A shows a single transducer module or panel 460 without a MEMS element. In the area other than the transducer array of the module 460, the module 460 is covered with a baffle. Thus, the baffle is behind each module in one embodiment. In one embodiment, the baffle is a rubber baffle in an irregular shape or highly attenuating material to absorb the acoustic energy and prevent the reverb echo. In contrast with FIG. 6, the baffle covers the area outside transducer to absorb the energy, but since there are more transducers the baffle material is generally behind or peripherally disposed around the module.

FIG. 16B shows a single transducer module or panel 460. Specifically, FIG. 16B is a view of a module with one row of transducers producing parallel beams of acoustic waves according to an illustrative embodiment of the invention.

FIG. 17 shows an exemplary progression of data collection over time with respect to a breast 260 as the body object of interest using a probe 480 such as for example the probe depicted in FIG. 15. Several panels or modules 260 are shown. Each module 490 has transducer elements or active elements 485. In one embodiment, the transducer elements are formed by cutting a unitary piezoelectric material or stacking layers of piezoelectric to form an array of square or rectangular elements. The circumferential groups of transducers are like the 1D probe array discussed above, but instead of individual loops or bands the circular transducers span multiple modules 490 as shown. Each module 490 is a 2D array while each circular row across multiple modules is a 1D array.

FIG. 18 shows a plurality of rectangular regions 500 associated with an ultrasound scan performed using a probe embodiment having a substantially cylindrical shape. As shown, composite image acquisition is performed for multiple slices. In one embodiment, the shorter side of each rectangle is substantially the same as the width of a given module. The image acquired from the transducer in one slice is composed of multiple rectangular regions obtained at different angles. This follows because the 2D arrays in each module are at different angles around the body object being scanned. As shown in FIG. 18, acoustic waves can be propagated within a rectangular volume that spans the diameter of the probe in length with the width corresponding to the width of a module. The acoustic waves are propagated from either end of the probe and are arranged in a substantially parallel configuration in one embodiment.

Since the same image areas are acquired from different angles, the composite image can be formed as a tomographic image similar to the process used in MRI image processing. With the image slice moving in z direction, a volume image dataset can be acquired for the 3D viewing. For a 15 cm diameter image slice, each segment image only needs to go slightly over 7.5 cm. A 7.5 MHz linear array probe is suitable for handling this level of tissue penetration. For the image acquired from each module, the system 140 performs the scan conversion with different angles and compounds the images together. In one embodiment, a wire target or phantom as shown below in FIG. 19 of known configuration can be placed within the container to provide a test pattern so that the system can calibrate itself for the scan converter algorithm. This scan converter algorithm or process collects the registration data due to a shift of the transducer modules. The wire target acquired from different modules after scan conversion should all display at the same spot on the screen. This allows the system to be calibrated.

To assure the images are aligned properly, a wire pattern phantom 530 as shown in FIG. 19 can be used to calibrate during the transducer module installation in the factory. The phantom is any dummy target to simulate the echo reflected from scanned object according to transmission of ultrasound waves. A cylinder wire cage can be used. During the calibration process, most of the registration mis-alignment is from the angular shifting in installation. After the initial installation, the angular shifting error can be corrected by scan converter algorithm with the wire calibration procedures. The wire targets are in a fixture for calibration as described above. The purpose of such calibration is mainly for adjusting the geometric error due to the assembly of the probe modules when operated together during the process of a scan conversion.

Again, it is to be understood that the embodiments described are for the purpose of illustration and are not intended to limit the invention specifically to those embodiments. For example, although the subject body part (e.g., breast) is shown as being immersed or submerged into an open container from the top, the container can also be placed onto a subject body part from any other direction. For example, the container can be placed onto a subject body part, and the container can have a conformal lip that conforms to the body part, and a seal can be made against the body part using any competent sealant, and then the container may be filled. Or, the container may be closed on its one otherwise-open end by a thin, flexible membrane, and the membrane can make contact with, and conform to the contours of, the subject body part, perhaps aided by an acoustic couplant or coupling solution to assure good acoustic coupling. Still other variations are within the scope of the present invention.

### Computer Embodiments

The present invention may be embodied in may different forms, including, but in no way limited to, computer program logic for use with a processor (e.g., a microprocessor, microcontroller, digital signal processor, or general purpose computer), programmable logic for use with a programmable logic device, (e.g., a Field Programmable Gate Array (FPGA) or other PLD), discrete components, integrated circuitry (e.g., an Application Specific Integrated Circuit (ASIC)), or any other means including any combination thereof. In a typical embodiment of the present invention, some or all of the processing of the data collected using a 1D or 2D ultrasound probe and the ultrasound system is implemented as a processor, control system, display, data input devices or other component and a set of computer program instructions that is converted into a computer executable form, stored as such in a computer readable medium, and executed by a microprocessor under the control of an operating system. Thus, transducer signals, control signals, and signal pulses are transformed into processor understandable instructions suitable for generating and collecting ultrasound data, generating dense tissue data, such as dense breast tissue data, generating three-dimensional images of a body object, performing tomographic processing and other embodiments and features described above.

Computer program logic implementing all or part of the functionality previously described herein may be embodied in various forms, including, but in no way limited to, a source code form, a computer executable form, and various intermediate forms (e.g., forms generated by an assembler, compiler, linker, or locator). Source code may include a series of computer program instructions implemented in any of various programming languages (e.g., an object code, an assembly language, or a high-level language such as Fortran, C, C++, JAVA, or HTML) for use with various operating systems or operating environments. The source code may define and use various data structures and communication messages. The source code may be in a computer executable form (e.g., via an interpreter), or the source code may be converted (e.g., via a translator, assembler, or compiler) into a computer executable form.

The computer program may be fixed in any form (e.g., source code form, computer executable form, or an intermediate form) either permanently or transitorily in a tangible storage medium, such as a semiconductor memory device (e.g., a RAM, ROM, PROM, EEPROM, or Flash-Programmable RAM), a magnetic memory device (e.g., a diskette or fixed disk), an optical memory device (e.g., a CD-ROM), a PC card (e.g., PCMCIA card), or other memory device. The computer program may be fixed in any form in a signal that is transmittable to a computer using any of various communication technologies, including, but in no way limited to, analog technologies, digital technologies, optical technologies, wireless technologies (e.g., Bluetooth), networking technologies, and internetworking technologies. The computer program may be distributed in any form as a removable storage medium with accompanying printed or electronic documentation (e.g., shrink-wrapped software), preloaded with a computer system (e.g., on system ROM or fixed disk), or distributed from a server over the communication system (e.g., the Internet or World Wide Web).

Hardware logic (including programmable logic for use with a programmable logic device) implementing all or part of the functionality previously described herein may be designed using traditional manual methods, or may be designed, captured, simulated, or documented electronically using various tools, such as Computer Aided Design (CAD), a hardware description language (e.g., VHDL or AHDL), or a PLD programming language (e.g., PALASM, ABEL, or CUPL).

Programmable logic may be fixed either permanently or transitorily in a tangible storage medium, such as a semiconductor memory device (e.g., a RAM, ROM, PROM, EEPROM, or Flash-Programmable RAM), a magnetic memory device (e.g., a diskette or fixed disk), an optical memory device (e.g., a CD-ROM), or other memory device. The programmable logic may be fixed in a signal that is transmittable to a computer using any of various communication technologies, including, but in no way limited to, analog technologies, digital technologies, optical technologies, wireless technologies (e.g., Bluetooth), networking technologies, and internetworking technologies. The programmable logic may be distributed as a removable storage medium with accompanying printed or electronic documentation (e.g., shrink-wrapped software), preloaded with a computer system (e.g., on system ROM or fixed disk), or distributed from a server over the communication system (e.g., the Internet or World Wide Web).

Various examples of suitable processing modules are discussed below in more detail. As used herein a module refers to software, hardware, or firmware suitable for performing a specific data processing or data transmission task. Typically, in a preferred embodiment a module refers to a software routine, program, or other memory resident application suitable for receiving, transforming, routing and processing instructions, or various types of data such as ultrasound scan data, transducer signal data, lesion boundary data, fatty tissue data, tumor data, acoustic wave data, tomographic data, analytic data, sets of two-dimensional scan data at different heights, cross-sectional or slice data relating to interior of body object, angular data, tissue data, tissue structure data, and other information of interest.

Computers and computer systems described herein may include operatively associated computer-readable media such as memory for storing software applications used in obtaining, processing, storing and/or communicating data. It can be appreciated that such memory can be internal, external, remote or local with respect to its operatively associated computer or computer system.

Memory may also include any means for storing software or other instructions including, for example and without limitation, a hard disk, an optical disk, floppy disk, DVD (digital versatile disc), CD (compact disc), memory stick, flash memory, ROM (read only memory), RAM (random access memory), DRAM (dynamic random access memory), PROM (programmable ROM), EEPROM (extended erasable PROM), and/or other like computer-readable media.

In general, computer-readable memory media applied in association with embodiments of the invention described herein may include any memory medium capable of storing instructions executed by a programmable apparatus. Where applicable, method steps described herein may be embodied or executed as instructions stored on a computer-readable memory medium or memory media. These instructions may be software embodied in various programming languages such as C++, C, Java, and/or a variety of other kinds of software programming languages that may be applied to create instructions in accordance with embodiments of the invention.

The aspects, embodiments, features, and examples of the invention are to be considered illustrative in all respects and are not intended to limit the invention, the scope of which is defined only by the claims. Other embodiments, modifications, and usages will be apparent to those skilled in the art without departing from the spirit and scope of the claimed invention.

The use of headings and sections in the application is not meant to limit the invention; each section can apply to any aspect, embodiment, or feature of the invention.

Throughout the application, where compositions are described as having, including, or comprising specific components, or where processes are described as having, including or comprising specific process steps, it is contemplated that compositions of the present teachings also consist essentially of, or consist of, the recited components, and that the processes of the present teachings also consist essentially of, or consist of, the recited process steps.

In the application, where an element or component is the to be included in and/or selected from a list of recited elements or components, it should be understood that the element or component can be any one of the recited elements or components and can be selected from a group consisting of two or more of the recited elements or components. Further, it should be understood that elements and/or features of a composition, an apparatus, or a method described herein can be combined in a variety of ways without departing from the spirit and scope of the present teachings, whether explicit or implicit herein.

The use of the terms “include,” “includes,” “including,” “have,” “has,” or “having” should be generally understood as open-ended and non-limiting unless specifically stated otherwise.

The use of the singular herein includes the plural (and vice versa) unless specifically stated otherwise. Moreover, the singular forms “a,” “an,” and “the” include plural forms unless the context clearly dictates otherwise. In addition, where the use of the term “about” is before a quantitative value, the present teachings also include the specific quantitative value itself, unless specifically stated otherwise.

It should be understood that the order of steps or order for performing certain actions is immaterial so long as the present teachings remain operable. Moreover, two or more steps or actions may be conducted simultaneously.

Where a range or list of values is provided, each intervening value between the upper and lower limits of that range or list of values is individually contemplated and is encompassed within the invention as if each value were specifically enumerated herein. In addition, smaller ranges between and including the upper and lower limits of a given range are contemplated and encompassed within the invention. The listing of exemplary values or ranges is not a disclaimer of other values or ranges between and including the upper and lower limits of a given range.

It is to be understood that the figures and descriptions of the invention have been simplified to illustrate elements that are relevant for a clear understanding of the invention, while eliminating, for purposes of clarity, other elements. Those of ordinary skill in the art will recognize, however, that these and other elements may be desirable. However, because such elements are well known in the art, and because they do not facilitate a better understanding of the invention, a discussion of such elements is not provided herein. It should be appreciated that the figures are presented for illustrative purposes and not as construction drawings. Omitted details and modifications or alternative embodiments are within the purview of persons of ordinary skill in the art.

It can be appreciated that, in certain aspects of the invention, a single component may be replaced by multiple components, and multiple components may be replaced by a single component, to provide an element or structure or to perform a given function or functions. Except where such substitution would not be operative to practice certain embodiments of the invention, such substitution is considered within the scope of the invention.

The examples presented herein are intended to illustrate potential and specific implementations of the invention. It can be appreciated that the examples are intended primarily for purposes of illustrative of the invention for those skilled in the art. There may be variations to these diagrams or the operations described herein without departing from the spirit of the invention. For instance, in certain cases, method steps or operations may be performed or executed in differing order, or operations may be added, deleted or modified.

Furthermore, whereas particular embodiments of the invention have been described herein for the purpose of illustrating the invention and not for the purpose of limiting the same, it will be appreciated by those of ordinary skill in the art that numerous variations of the details, materials and arrangement of elements, steps, structures, and/or parts may be made within the principle and scope of the invention without departing from the invention as described in the claims.

I claim:

1. An ultrasonic image scanning system for scanning a body object having one or more tissue structures disposed therein comprising:
an immersible ultrasound probe having a substantially cylindrical shape, the probe comprising
an inner surface defining a cavity having at least one opening sized to receive the body object, the inner surface configured to receive acoustic signals while immersed in a fluid; and
a plurality of modules arranged to form the inner surface, each module comprising a high voltage multiplexer, and a plurality of ultrasonic transducers, wherein the plurality of ultrasonic transducers is arranged in a plurality of rows and columns, wherein each row is adjacent to at least another row.

2. The ultrasonic image scanning system of claim 1 further comprising a baffle layer positioned in each module behind the plurality of ultrasonic transducers to reduce an acoustic reverberation associated with ultrasonic image scanning.

3. The ultrasonic image scanning system of claim 1 wherein each module of the plurality of modules has a substantially rectangular shape having a length that ranges from about 100 mm to about 300 mm and a width that ranges from about 30 mm to about 60 mm.

4. The ultrasonic image scanning system of claim 1 wherein the plurality of transducers per module is at least 64 transducer elements.

5. The ultrasonic image scanning system of claim 1 wherein each of the plurality of ultrasonic transducers comprises a material selected from the group consisting of Lead zirconate titanate, a ceramic PZT material, CMUT, PMUT, and a semiconductor.

6. The ultrasonic image scanning system of claim 1 wherein each of the plurality of ultrasonic transducers is a MEMS device configured to produce substantially parallel beams on a per row basis and in electronic communication with at least one of the high voltage multiplexers.

7. The ultrasonic image scanning system of claim 1 wherein high voltage multiplexer is in electrical communication with the probe and configured to route signals between the probe and an ultrasound system.

8. The ultrasonic image scanning system of claim 1 further comprising a computer aided diagnostic system for processing a set of two-dimensional or three-dimensional image scans to display tissue structural characteristic details of the body object and identify one or more tissue structures.

9. The ultrasonic image scanning system of claim 1 wherein each module comprises a flexible or rigid printed circuit board configured to support each of the ultrasonic transducers and orient them towards the body object.

10. The ultrasonic image scanning system of claim 1 further comprising a container configured to receive the probe, the container comprising a port for filling the container with coupling solution.

11. The ultrasonic image scanning system of claim 1 wherein the probe is configured to prevent excessive tissue heating during a scan such that the temperature of the body object ranges from 37 degrees Celsius to about 42 degrees Celsius during a scan.

12. The ultrasonic image scanning system of claim 1 wherein the probe has a volume scan rate that ranges from about 0.5 volumes per second to about 50 volumes per second.

13. The ultrasonic image scanning system of claim 1 further comprising a control system in electrical communication with the probe and configured to trigger frame capture and transmit a clock pulse to one of the MEMS or the multiplexer.

14. A method for ultrasonically scanning a body object having one or more tissue structures disposed therein comprising:
electronically addressing a first plurality of transducers arranged in substantially circular configuration using a first control signal;
transmitting a first plurality of incident acoustic waves from the first plurality of transducers in response to the first control signal;
receiving a first plurality of returning acoustic waves at the first plurality of transducers, the first plurality of returning acoustic waves reflected from a body object;
electronically addressing a second plurality of transducers arranged in substantially circular configuration using a second control signal;
transmitting a second plurality of incident acoustic waves from the second plurality of transducers in response to the second control signal; and
receiving a second plurality of returning acoustic waves at the second plurality of transducers, the second plurality of returning acoustic waves reflected from the body object.

15. The method of claim 14 further comprising the steps of converting the first plurality of returning acoustic waves and the second plurality of returning acoustic waves into one or more electrical signals and generating an image of the body object using an ultrasound system and the one or more electrical signals.

16. The method of claim 14 wherein the body object is a breast or any soft tissue and further comprising the step of tomographically rendering a three-dimensional image of the breast or any soft tissue and one or more tissue structures disposed therein.

17. The method of claim 14 further comprising the step of heating the body object such that the change in a temperature of the body object is less than between about 37 and about 42 degrees Celsius.

18. The method of claim 14 further comprising the step of generating a tomographic image of the body object using the first and second pluralities of returning acoustic waves.

19. The method of claim 18 further comprising the step of displaying one or more tissue structures.

20. The method of claim 14 wherein the steps of the method are performed within between about 1 second to about 10 seconds.

21. The method of claim 14 further comprising the step of identifying a tissue structure as likely to be cancerous on a display.

22. The method of claim 14 further comprising the step of identifying a tissue structure as likely to be benign on a display.

23. The method of claim 14 wherein the first plurality of incident acoustic waves is arranged in a substantially parallel configuration.

24. The method of claim 23 wherein the second plurality of incident acoustic waves is arranged in a substantially parallel configuration.
